(19) United States
(12) Patent Application Publication
(10) Pub. No.: US 2017/0007459 A1
(43) Pub. Date: Jan. 12, 2017

(54) VISION AIDING METHOD AND APPARATUS INTEGRATED WITH A CAMERA MODULE AND A LIGHT SENSOR

(71) Applicant: QINGDAO GOERTEK TECHNOLOGY CO., LTD., Shandong (CN)

(72) Inventors: Xuebing Deng, Qingdao City (CN); Jiantang Gong, Qingdao City (CN)

(21) Appl. No.: 15/115,111

(22) PCT Filed: Jun. 29, 2015
(86) PCT No.: PCT/CN2015/082633

§ 371 Date: Jul. 28, 2016

(30) Foreign Application Priority Data
Jun. 30, 2014 (CN) 201410304779.6

(51) Int. Cl. A61F 9/08; A61F 4/00; H04N 5/225

(52) U.S. Cl. CPC A61F 9/08; H04N 5/2257; H04N 5/2256; A61F 4/00

(57) ABSTRACT

A vision aiding method employing a camera and a light sensor includes detecting data of ambient light intensity by the light sensor and giving a user a first kind of visual aids when the detected ambient light intensity exceeds a first preset value for a set period of time, giving the user a second kind of visual aids when the detected ambient light intensity is below a second preset value for a set period of time, and in the process of the second kind of visual aids, starting the camera to acquire image data of surrounding environment of the user when it is detected that the ambient light intensity changes for a certain period of time, and giving the user a third kind of visual aids when it is determined that there is a vehicle passing according to the image data of the surrounding environment and the ambient light intensity.

### CROSS-REFERENCE TO RELATED APPLICATION

This application is a U.S. National-Stage entry under 35 U.S.C. §371 based on International Application No. PCT/CN2015/082633, filed Jun. 29, 2015 and which claims priority to Chinese Application No. 201410304779.6, filed Jun. 30, 2016, which are all hereby incorporated in their entirety by reference.

### TECHNICAL FIELD

This application pertains to an apparatus for aiding vision, particularly to a vision aiding method and apparatus integrated with a camera module and a light sensor.

### BACKGROUND

It is well known that, eye diseases have been one of the major problems of the health field. According to the international and domestic statistics, one in five persons with disabilities involves visual disability. Humans acquire information primarily through vision. Therefore, the visual disability is one of the most severe and most painful disabilities of the disabled. Domestically and abroad, ultrasonic or infrared probes are usually used to find obstacles so as to help the blind, however, the effects of which are poor. Some similar high-grade products are developed toward a robot, the costs of which, however, due to the complex mechanical structure of the robot and the high-grade artificial intelligence development, remain high and which are difficult to popularize.

With the development of science and technology, electronic elements such as a camera module and a sensor, etc. are applied more and more to a variety of scenarios, the use schemes have also become increasingly mature, and the help for people is also growing. In the meanwhile, in order that the persons with the visual disabilities share the innovation brought by the new technology better, products for the persons with the visual disabilities are also gradually increasing. Equipments like readers for the blind, positioning devices for the blind and etc. emerge one after another. However, equipments for real-time detection and judgment on walking environment, for reminding of vehicles in the front and rear etc. are still absent. There are still great safety risks when the blind go outside alone.

Therefore, an apparatus for aiding vision is needed, in order to avoid the above defects.

In addition, other objects, desirable features and characteristics will become apparent from the subsequent summary and detailed description, and the appended claims, taken in conjunction with the accompanying drawings and this background.

### SUMMARY

An object of the present invention is to provide a vision aiding method and apparatus integrated with a camera module and a light sensor, which can carry out visual aids for users and perform voice prompts so as to protect the personal safety of the users.

The present invention provides a vision aiding method integrated with a camera module and a light sensor, comprising the following steps:

a. detecting data of ambient light intensity by the light sensor in real time, and giving a user a first kind of visual aids when the detected ambient light intensity is consistently higher than a first preset value for a set period of time;

b. giving the user a second kind of visual aids when the detected ambient light intensity is consistently lower than a second preset value for a set period of time;

c. in the process of the second kind of visual aids, starting the camera module to acquire image data of surrounding environment of the user when it is further detected that the ambient light intensity changes consistently for a certain period of time, and giving the user a third kind of visual aids when it is determined that there is vehicle passing according to the image data of the surrounding environment and the ambient light intensity.

Further, in the process of the first kind of visual aids and in the process of the second kind of visual aids, the user can turn on the camera module through a manual switch at any time to acquire image data of the surrounding environment or turn off the camera module; and the user is given a fourth kind of visual aids according to the image data of the surrounding environment and the ambient light intensity, the fourth kind of visual aids being incorporated in the process of the first kind of visual aids and in the process of the second kind of visual aids.

Further, the process of the first kind of visual aids is accompanied with a first kind of prompt, the process of the second kind of visual aids is accompanied with a second kind of prompt, the process of the third kind of visual aids is accompanied with a third kind of prompt, and the process of the fourth kind of visual aids is accompanied with a fourth kind of prompt, wherein the first kind of prompt, the second kind of prompt, the third kind of prompt and the fourth kind of prompt adopt one or more of a voice prompt, a vibrating prompt and a LED light flashing prompt.

Further, when the determination fails, the user can be prompted that the determination fails.

The present invention further provides a vision aiding apparatus integrated with a camera module and a light sensor, comprising:

at least one light sensor for detecting data of ambient light intensity in real time;

a camera module for photographing videos or images of surrounding environment;

a processing chip for receiving the data from the light sensor and the camera module, and giving a user a first kind of visual aids after determining that the ambient light intensity has been consistently higher than a first preset value for a set period of time; giving the user a second kind of visual aids when determining that the ambient light intensity has been consistently lower than a second preset value for a set period of time; and starting the camera module to acquire image data of the surrounding environment of the user when further determining that the ambient light intensity changes consistently for a certain period of time in the process of the second kind of visual aids, and giving the user a third kind of visual aids when determining that there is a vehicle passing according to the image data of the surrounding environment and the ambient light intensity.

Further, the apparatus further comprises at least one location indicating module connected to the processing chip, for indicating location of the user to crowd in the surrounding environment under control of the processing chip. The location indicating module can indicate the location of the user to crowd in the surrounding environment by voice, a LED light or vibrating.

Further, the apparatus further comprises a second switch for manually turning on and off the camera module so as to acquire image data of the surrounding environment at any time in the process of the first kind of visual aids and the process of the second kind of visual aids, and give the user a fourth kind of visual aids according to the image data of the surrounding environment and the ambient light intensity, the fourth kind of visual aids being incorporated in the process of the first kind of visual aids and in the process of the second kind of visual aids.

Further, the apparatus further comprises a prompting module connected to the processing chip, for under control of the processing chip, for the user, performing a first kind of prompt in the process of the first kind of visual aids, performing a second kind of prompt in the process of the second kind of visual aids, performing a third kind of prompt in the process of the third kind of visual aids, performing a fourth kind of prompt in the process of the fourth kind of visual aids, wherein the first kind of prompt, the second kind of prompt, the third kind of prompt and the fourth kind of prompt adopt one or more of a voice prompt, a vibrating prompt and a LED light flashing prompt.

Further, the light sensor comprises a front light sensor and a rear light sensor which can sense light conditions in front and rear of the user and transmit signals to the processing chip; or the light sensor comprises a front light sensor, a rear light sensor, a left light sensor and a right light sensor which can sense the light conditions in front and rear of, on the left side of and on the right side of the user and transmit signals to the processing chip.

Further, the apparatus further comprises a light source module, the light source comprises a front light source module and/or a rear light source module for illuminating roads, assisting the camera module in photographing operations in dark environment.

Further, the apparatus further comprises a GPS module connected to the processing chip, for locating geographical location of the user and feeding back the geographical location to the processing chip, the processing chip confirming the surrounding environment of the user in combination with the geographical location and the data photographed by the camera module, to assist navigation for the user.

In the visual aids for the user, common assistant prompting methods comprises: alerting passers-by by LED light flashing, prompting the user by vibrator vibrating and prompting the user by voice. Wherein the contents of the voice prompting can include: in dark, please warn; traffic light ahead, please pay attention; red light, stop please; green light, go ahead please; downstairs ahead, please pay attention; upstairs ahead, please pay attention; a car is coming, please to the right; a car is coming, please to the left; turn left ahead, please pay attention; turn right ahead, please pay attention; escalator ahead, please pay attention; tunnel ahead, please pay attention; and so on, but not limited to these.

According to the present invention, when the brightness of the ambient light detected by the light sensor is lower than a preset value, the processing chip determines that it is a dark state such as in a tunnel, in a underpass or at night, informs the user of being in the dark environment state through the prompting module and controls the location indicating module to alert by LED light flashing so as to make the passers-by pay attention to avoid the user. After the data detected by the light sensor in real time has risen to the preset value or above for a set time, the location indicating module is closed, and the apparatus actively turn on the camera module to photograph the road conditions. If the processing chip determines that the sudden change of the light intensity results from the change of fixed facilities by comparing the images, the location indicating module is turned on to prompt the user about the location. If it does not result from the change of fixed facilities, it is considered that the sudden change of the light intensity results from a vehicle passing, and data sources and the change of the light intensity are analyzed, the location of a coming vehicle is determined and an approximate distance is inferred. Then, according to the images captured by the camera module, the user is alerted by vibrating of the vibrator and prompted about the direction of dodge and how to avoid by voice from the voice prompting module. The present invention can determine in time and effectively that the conditions of the road ahead is street, sidewalk, bushes or the like, and prompt the user about the road conditions. The conditions such as the road intersections or the traffic lights can be further determined. At night or inside the tunnel, the vehicles in the front and rear can also be determined by the built-in front and rear light sensors so as to alert the user, and the direction of dodge can be prompted by voice from the voice prompting module, and the crowd in the surrounding environment can be alerted by the LED light flashing to pay attention to the person with visual disabilities, that is to say, the present invention not only can prompt the user in real time, but also can alert the persons around, which provide a guarantee for the personal safety of the user.

Other features and advantages of the present application will be set forth in the following description, and will become obvious partly from the description, or will be understood by carrying out the present application. The object and other advantages of the present application can be realized and obtained through the structures pointed out specifically in the written description, the claims and the attached drawings.

### BRIEF DESCRIPTION OF DRAWINGS

The present invention will hereinafter be described in conjunction with the following drawing figures, wherein like numerals denote like elements, and:

FIG. 1 is a method flowchart illustrating a non-limiting embodiment of the vision aiding method integrated with a camera module and a light sensor according to the present invention;

FIG. 2 is a block diagram illustrating a non-limiting Embodiment 1 of the vision aiding apparatus integrated with a camera module and a light sensor according to the present invention;

FIG. 3 is a flowchart illustrating a non-limiting embodiment of the operation mode of the light sensor of the Embodiment 1 of the present invention;

FIG. 4 is a flowchart illustrating a non-limiting embodiment of the operation mode of the camera module of the Embodiment 1 of the present invention;

FIG. 5 is a block diagram of Embodiment 2 of the vision aiding apparatus integrated with a camera module and a light sensor according to a non-limiting embodiment of the present invention.

Reference signs: 11 processing chip, 21 front light sensor, 22 front light source module, 32 voice prompting module, 33 front/rear indicating LEDs, 34 vibrator, 41 camera module, 42 second switch, 43 GPRS module, 51 rear light sensor, 52 rear light source module.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

To make the objects and features of the present invention clearer and more understandable, specific implementations of the present invention will be further explained in connection with the drawings below. However, the present invention can be implemented in different forms and should not be considered to be only limited to the described embodiments.

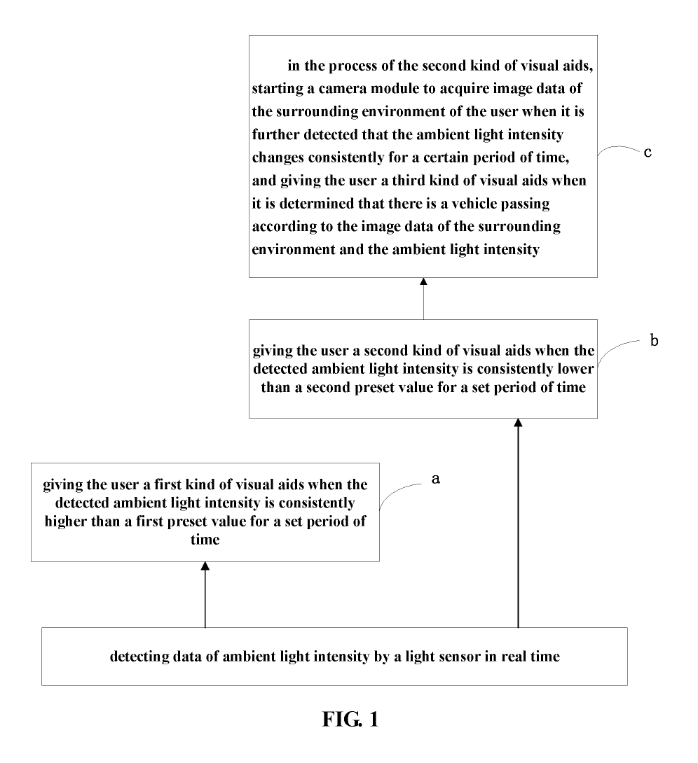

As shown in FIG. 1, the present embodiment provides a vision aiding method integrated with a camera module and a light sensor, which comprises the following steps:

a. detecting, by the light sensor, data of ambient light intensity in real time, and giving a user a first kind of visual aids when the detected ambient light intensity is consistently higher than a first preset value for a set period of time;

b. giving the user a second kind of visual aids when the detected ambient light intensity is consistently lower than a second preset value for a set period of time;

c. in the process of the second kind of visual aids, starting the camera module to acquire image data of the surrounding environment of the user when it is further detected that the ambient light intensity changes consistently for a certain period of time, and giving the user a third kind of visual aids when it is determined that there is a vehicle passing according to the image data of the surrounding environment and the ambient light intensity.

The specific principle is as follows: When a front and a rear light sensors detect that the ambient light is lower than a preset value, a processing chip determines that the environment where the user is located is a dark environment such as in a tunnel, in a underpass or in a black day. The processing chip turns on a front and a rear indicating LEDs to flash for alterting and informs, in combination with a voice prompting module, the user that he/she is currently located in a dark environment. The prompting module will be closed after the data detected by the front and rear light sensors in real time has risen to a preset value or above for a set period of time. In addition, when any of the light sensors detects that the light intensity changes suddenly to a set state within a preset time, the apparatus will start the camera module actively to photograph road conditions, and the processing chip can determine, by comparing images, whether the sudden change of the light intensity results from changes of fixed facilities (such as entering a tunnel, a underpass or being obscured by a foreign object, etc.) or not (such as a vehicle passing). If it results from the changes of fixed facilities, a location indicating module will be turned on to prompt location. If it does not result from the changes of fixed facilities, it is considered that the sudden change of the light intensity results from a vehicle passing, and data sources and an amount of the change of the light intensity are analyzed, the location of a coming vehicle is determined and an approximate distance is inferred. Then, according to the images captured by the camera module, the user is alerted by vibrating and prompted about the direction of dodge and how to avoid by voice. Because there are light sensors in the front and at the back, the direction of the coming vehicle can be determined. Furthermore, because an algorithm for the change of the light intensity per a unit of time is adopted, the case of determination failure resulting from non-front placement or angle offset can be avoided. Further, because a light intensity sensor performs real-time detection, if it is found that the light intensity has been lower than a very low value for more than a certain period of time, it is considered that it is obscured by a foreign object and at this time it will also prompt by vibrating and voice, so as to prevent the failure of the determination due to being obscured.

When a person with the visual disabilities needs to determine the road conditions (for example, cases in which there are no lanes for the blind or obstacles stairs are encountered), he/she can turn on a switch of the camera module, and the front light sensor detects the ambient light intensity and determines whether it is needed to turn on a light source module to assist the photographing. Then, the camera module performs the photographing and sends photographs back to the processing chip. The processing chip determines, by comparing the photographs sent back with the images in an image library, which kind of road conditions is in front of the user. If it is an intersection of roads, then the processing chip further determines whether it is a red light or a green light. After the determination is complete, a vibrator will be controlled to alert by vibrating and prompt the user by voice. Then, the user will be guided by the voice prompting module to act accordingly.

### Embodiment 1

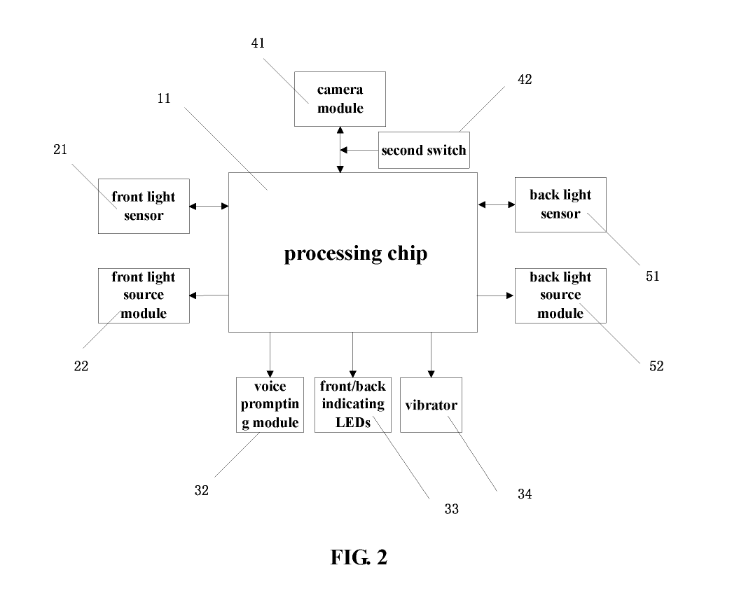

As shown in FIG. 2, the present embodiment provides a vision aiding apparatus integrated with a camera module and a light sensor, including:

a light sensor for detecting data of ambient light intensity in real time;

a camera module 41 for photographing videos or images of the surrounding environment;

a processing chip 11 for receiving the data from the light sensor and the camera module 41, and giving a user a first kind of visual aids including informing the user that it is currently day time or in a bright area, when it is determined that the ambient light intensity has been consistently higher than a first preset value for a set period of time; for giving the user a second kind of visual aids including informing the user that it is currently a dark day or in a dark environment or he/she goes into a tunnel and so on, when it is determined that the ambient light intensity has been consistently lower than a second preset value for a set period of time; and starting the camera module 41 to acquire image data of the surrounding environment of the user when further determining that the ambient light intensity changes consistently for a certain period of time in the process of the second kind of visual aids, and giving the user a third kind of visual aids including informing the user of being in a darker environment, there being vehicles approaching and paying attention to dodge and to which direction to dodge, etc., when determining that there is a vehicle passing according to the detected surrounding environment and ambient light intensity.

The light sensor comprises a front light sensor 21 and a rear light sensor 51, which can sense light conditions in front and rear of the user and transmit signals to the processing chip 11. Alternatively, the light sensor comprises the front light sensor 21, the rear light sensor 51, a left light sensor and a right light sensor, which can sense the light conditions in front and rear of, on the left and right sides of the user and transmit signals to the processing chip 11.

The apparatus further comprises a light source module which comprises a front light source module 22 and a rear light source module 52. In the present embodiment, the light source module adopts LED lights for illuminating the road and assisting the camera module 41 in photographing operations in the dark environment.

In the present embodiment, the apparatus further comprises a second switch 42 for manually turning on and off the camera module 41, for acquiring image data of the surrounding environment at any time in the process of the first and second kinds of visual aids, and giving the user a fourth kind of visual aids in accordance with the image data of the surrounding environment and the ambient light intensity, the fourth kind of visual aids being integrated in the process of the first and second kinds of visual aids.

In the present embodiment, the apparatus further comprises a prompting module connected to the processing chip 11. The prompting module comprises a voice prompting module 32, a front/rear indicating LED 33 and a vibrator 34, and perform, for the user under the control of the processing chip 11: a first kind of prompt in the process of the first kind of visual aids, in which the user is informed, by the vibrating of the vibrator 34 in cooperation with the voice prompting module 32, that it is currently daytime or he/she is located in a bright environment, and the user can be guided by voice from the voice prompting module 32 in combination with the photographing results of the camera module 41, so as to assist the user to walk; a second kind of prompting in the process of the second kind of visual aids, in which the persons around the user is alerted by flashing of the indicating LEDs, so as to make the passers-by pay attention to and avoid the user; a third kind of prompt in the process of the third kind of visual aids, in which coming vehicles are determined according to the change of the surrounding ambient light intensity detected by the front light sensor 21 and/or the rear light sensor 51, the front light source module 22 and/or the rear light source module 52 and the camera module 41 are started to photograph the road conditions around, and the user is prompted by the vibrator 34 and is guided how to dodge by means of the voice prompting module 32; and a fourth kind of prompt in the process of the fourth kind of visual aids, in which the user is guided by turning on the light source module in combination with the camera module.

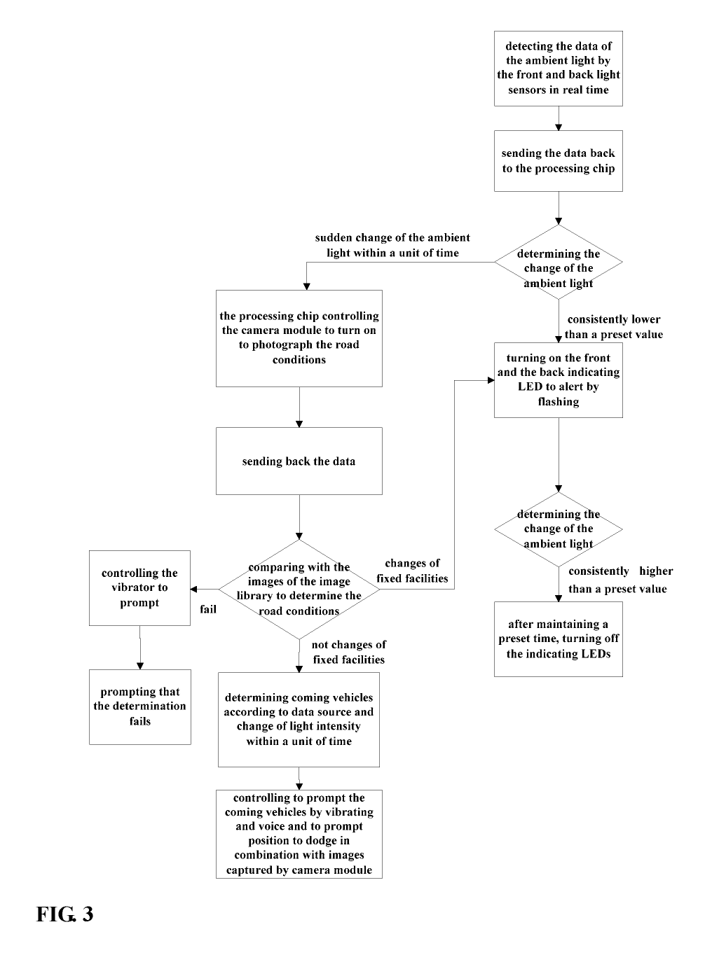

As shown in FIG. 3, FIG. 3 is a flowchart of the operation mode of the light sensor of the Embodiment 1 of the present invention, which shows the work flow of the light sensor. After the front light sensor 21 or the rear light sensor 51 of the apparatus detects the data of the ambient light in real time, the data is transferred synchronously to the processing chip 11 of the apparatus. The processing chip 11 determines the change of the ambient light. When the ambient light is consistently lower than a preset value, the apparatus turns on the front and rear indicating LEDs 33 which flash to alert the persons around, so that the passers-by around can dodge and avoid the user. When the processing chip 11 detects a sudden change of the ambient light within a unit of time, the processing chip 11 turns on the camera module 41 to photograph the road conditions. The photographed data of the road conditions is sent back to the processing chip 11. The processing chip 11 judges the road conditions by comparing the data of the road conditions sent back by the camera module 41 with images in the image library. If the sudden change of the ambient light results from the changes of fixed facilities, the front and rear indicating LEDs 33 are turned on to alert the persons around by flashing. If it does not result from the changes of fixed facilities, it is determined, through the data sources and the change of the light intensity within a unit of time, that there are coming vehicles, then the processing chip 11 controls the vibrator 34 to vibrate, and alerts the user to the coming vehicles through the voice prompting module and prompts the user to dodge and avoid in combination with the camera module 41. If the comparison of images fails, the processing chip 11 controls the vibrator 34 to vibrate to alert the user to the failure of the determination.

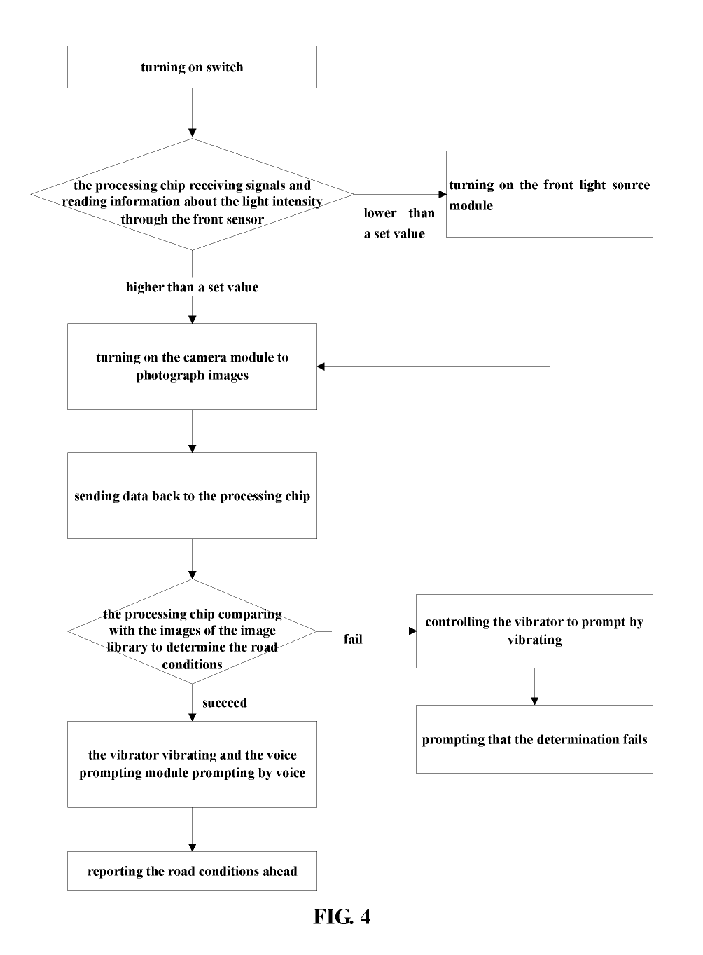

As shown in FIG. 4, FIG. 4 is a flowchart of the operation mode of the camera module 41 of the Embodiment 1 of the present invention. In an operating state of the apparatus, the processing chip 11 receives signals and reads information of the light intensity through the front light sensor 21. When the light intensity is higher than a preset value, the camera module 41 is turned on to photograph images. When the light intensity is lower than the preset value, the processing chip 11 controls to turn on the front light source module 22 and/or the rear light source module 52 and then turn on the camera module 41 to photograph images. The camera module 41 sends the data of the photographed images back to the processing chip 11. The processing chip 11 performs determination by comparing the images sent back by the camera module 41 with the images in the image library. If the determination fails, the processing chip 11 controls the vibrator 34 to vibrate so as to alert the user to the determination results. If the determination succeeds, the processing chip 11 controls the vibrator 34 to vibrate and inform the user of the road conditions in combination with the voice prompting module 32.

According to the present invention, when the brightness of the ambient light detected by the light sensors is lower than a preset value, the processing chip 11 determines that it is a dark state such as in a tunnel, in a underpass or at night, and turns on the front indicating LED and/or the rear indicating LED to alert by flashing. After the data detected by the light sensor in real time has risen to the preset value or above for a set time, the front/rear indicating LEDs 33 will be closed. The apparatus will actively turn on the camera module 41 to photograph the road conditions. If the processing chip 11 determines that the sudden change of the light intensity results from the changes of fixed facilities by comparing the images, the front indicating LED and/or the rear indicating LED are/is turned on to prompt the user about the location. If it does not result from the changes of fixed facilities, it is considered that the sudden change of the light intensity results from a vehicle passing, and data sources and the change of the light intensity are analyzed, the location of a coming vehicle is determined and an approximate distance is inferred. Then, according to the images captured by the camera module 41, the user is alerted by vibrating and prompted to dodge and avoid by voice. According to the embodiment of the present invention, it can be determined in time and effectively that the conditions of the road ahead are street/sidewalk/bushes, etc. and the prompt about the road conditions can be performed. The conditions such as the road intersections or the traffic lights can be further determined. At night or inside the tunnel, the vehicles in the front and at the back can also be determined by the built-in front and rear light sensor and alert can be perform by vibrating, and the persons around can be prompted by the front indicating LED and/or the rear indicating LED flashing to pay attention to the person with visual disabilities, which not only can prompt the user in real time, but also can alert and indicate the persons around, so as to provide a guarantee for the personal safety of the user.

It should be noted that, the apparatus of the present embodiment can further be divided into two modes: a light sensing mode and an imaging mode.

The light sensing mode is in a normally on state, namely the light sensor is in the operating state in the processes of the first kind of visual aids, the second kind of visual aids, the third kind of visual aids and the fourth kind of visual aids. For example, when the ambient light detected by the front light sensor 21 and/or the rear light sensor 51 is lower than a set value, the processing chip 11 can determine that the surrounding environment of the user is in a weak light state such as in a tunnel, in a underpass or in a dark day, then the front and rear indicating LEDs will be turned on to alert by flashing. After the data detected by the front light sensor 21 and/or the rear light sensor 51 in real time maintained at a set value or lower for a certain period of time, the indicating LEDs will be closed. In addition, in an environment with weak light, when light intensity detected by any of the light sensors consistently changes within a preset time, the processing chip 11 actively turns on the camera module 41 to photograph the surrounding environment (for example the road conditions). By comparing the images, it can be determined whether the reason of the change of the light intensity is changes of fixed facilities (such as entering a tunnel, a underpass or being obscured by a foreign object, etc.) or not (such as a vehicle passing). If it results from the changes of fixed facilities, the indicating LEDs will be turned on to prompt about the location. If it does not result from the changes of fixed facilities, it will be determined that a vehicle is passing, and data sources and an amount of the change of the light intensity will be analyzed, the location of a coming vehicle will be determined and an approximate distance will be inferred. Then, the user will be alerted by vibrating and prompted by voice to dodge, and the user will be prompted about the avoiding direction in combination with the images of the camera module 41. Because there are light sensors both in the front and at the back, the direction of the coming vehicle in the front and at the back can be determined. Furthermore, an algorithm for the change of the light intensity per a unit of time can be adopted so as to avoid determination failure resulting from non-front placement and angle offset. Further, the light sensors perform real-time detection. If it is found that the light intensity has been lower than a very low value for more than a certain period of time, it is considered that it is obscured by a foreign object and at this time vibrator 34 will vibrate and the voice prompting module 32 will prompt by voice so as to prevent the failure of the determination due to being obscured.

The imaging mode is a triggered mode. The camera module 41 can be automatically triggered by the processing chip 11 as necessary, and can also be manually triggered based on the subjective needs of the user, thereby power consumption of the apparatus can be reduced. For example, when the user subjectively needs to determine the road conditions (for example, needs to determine the conditions of the traffic lights, whether there are lanes for the blind or whether the user encounters obstacles such as stairs), the manual switch can be pressed. At this time, the front light sensor detects the ambient light intensity and determines whether it needs to turn on the LED light source to assist photographing. Then, the camera module 41 performs photographing and sends the photographs back to the processing chip 11. The processing chip 11 determines which kind of condition the image ahead is by comparing with the image library, for example, determines it is a red light or a green light at a road intersection, and after the determination is complete, controls the vibrator 34 to prompt the user by vibrating that a voice prompt will be performed. Then, the condition of the traffic light will be reported by the voice prompting module 32.

### Embodiment 2

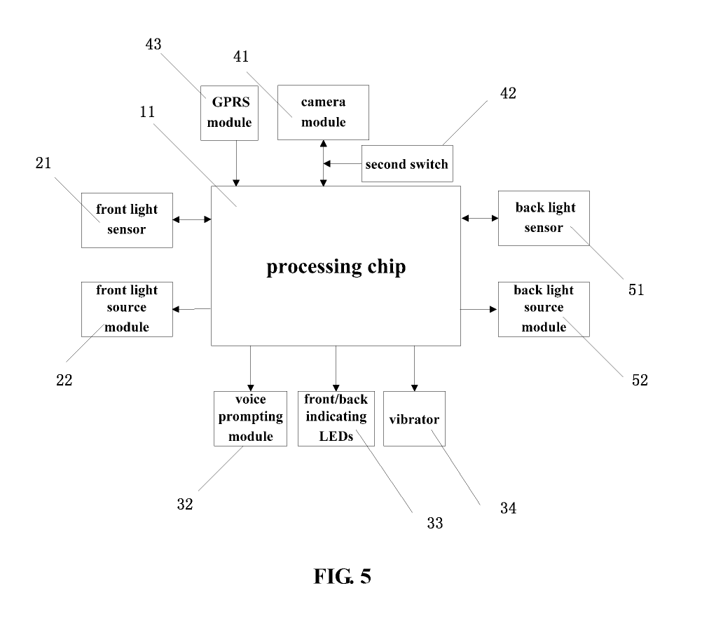

As shown in FIG. 5, the present embodiment is different from the Embodiment 1 in that the processing chip 11 is further connected to a GPS module.

The present embodiment can confirm the road conditions in real time and the changes of the environment facilities more accurately in combination with the camera module 41 and can navigate the user in combination with the voice prompting module 32, which not only guarantees the personal safety of the use on the road, but also can guide the user to find the way home, so as to avoid the situation that he/she cannot go home because of getting lost.

In other embodiments of the present invention, the apparatus further comprises a clock chip. The processing chip 11 can collect information about the clock and report the time by voice to the user according to the information about the clock and determine whether it is currently day or night in combination with the information about the clock.

In other embodiments of the present invention, the apparatus further comprises a weather forecast module. The weather forecast module updates the weather forecast through the WIFI, the mobile communication network and etc.. The processing chip 11 can collect information about the weather forecast, inform the user of the weather by voice according to the information about the weather forecast and determine whether it is currently sunny or cloudy in combination with the information about the weather forecast.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. A vision aiding method integrated with a camera module and a light sensor, comprising the following steps:
a. detecting data of ambient light intensity by the light sensor in real time, and giving a user a first kind of visual aids when the detected ambient light intensity is consistently higher than a first preset value for a set period of time;
b. giving the user a second kind of visual aids when the detected ambient light intensity is consistently lower than a second preset value for a set period of time;
c. in the process of the second kind of visual aids, starting the camera module to acquire image data of surrounding environment of the user when it is further detected that the ambient light intensity changes consistently for a certain period of time, and giving the user a third kind of visual aids when it is determined that there is a vehicle passing according to the image data of the surrounding environment and the ambient light intensity.

2. The vision aiding method integrated with the camera module and the light sensor according to claim 1, further comprising: in the process of the first kind of visual aids and in the process of the second kind of visual aids, turning on the camera module through a manual switch by the user at any time to acquire image data of the surrounding environment or turning off the camera module; and giving the user a fourth kind of visual aids according to the image data of the surrounding environment and the ambient light intensity, the fourth kind of visual aids being incorporated in the process of the first kind of visual aids and in the process of the second kind of visual aids.

3. The vision aiding method integrated with the camera module and the light sensor according to claim 2, wherein the process of the first kind of visual aids is accompanied with a first kind of prompt, the process of the second kind of visual aids is accompanied with a second kind of prompt, the process of the third kind of visual aids is accompanied with a third kind of prompt, and the process of the fourth kind of visual aids is accompanied with a fourth kind of prompt, wherein the first kind of prompt, the second kind of prompt, the third kind of prompt and the fourth kind of prompt adopt one or more of a voice prompt, a vibrating prompt and a LED light flashing prompt.

4. A vision aiding apparatus integrated with a camera module and a light sensor, comprising:
at least one light sensor for detecting data of ambient light intensity in real time;
a camera module for photographing videos or images of surrounding environment;
a processing chip for receiving the data from the light sensor and the camera module, and giving a user a first kind of visual aids after determining that the ambient light intensity has been consistently higher than a first preset value for a set period of time; giving the user a second kind of visual aids when determining that the ambient light intensity has been consistently lower than a second preset value for a set period of time; and starting the camera module to acquire image data of the surrounding environment of the user when further determining that the ambient light intensity changes consistently for a certain period of time in the process of the second kind of visual aids, and giving the user a third kind of visual aids when determining that there is a vehicle passing according to the image data of the surrounding environment and the ambient light intensity.

5. The vision aiding apparatus integrated with the camera module and the light sensor according to claim 4, the apparatus further comprises at least one location indicating module connected to the processing chip, for indicating location of the user to crowd in the surrounding environment under control of the processing chip.

6. The vision aiding apparatus integrated with the camera module and the light sensor according to claim 4, the apparatus further comprises a second switch for manually turning on and off the camera module so as to acquire image data of the surrounding environment at any time in the process of the first kind of visual aids and the process of the second kind of visual aids, and give the user a fourth kind of visual aids according to the image data of the surrounding environment and the ambient light intensity, the fourth kind of visual aids being incorporated in the process of the first kind of visual aids and in the process of the second kind of visual aids.

7. The vision aiding apparatus integrated with the camera module and the light sensor according to claim 6, the apparatus further comprises a prompting module connected to the processing chip, for under control of the processing chip, for the user, performing a first kind of prompt in the process of the first kind of visual aids, performing a second kind of prompt in the process of the second kind of visual aids, performing a third kind of prompt in the process of the third kind of visual aids, performing a fourth kind of prompt in the process of the fourth kind of visual aids, wherein the first kind of prompt, the second kind of prompt, the third kind of prompt and the fourth kind of prompt adopt one or more of a voice prompt, a vibrating prompt and a LED light flashing prompt.

8. The vision aiding apparatus integrated with the camera module and the light sensor according to claim 4, the light sensor comprises a front light sensor and a rear light sensor which can sense light conditions in front and rear of the user and transmit signals to the processing chip; or the light sensor comprises a front light sensor, a rear light sensor, a left light sensor and a right light sensor which can sense the light conditions in front and rear of, on the left side of and on the right side of the user and transmit the signals to the processing chip.

9. The vision aiding apparatus integrated with the camera module and the light sensor according to claim 4, the apparatus further comprises a light source module, the light source comprises a front light source module and/or a rear light source module for illuminating roads, assisting the camera module in photographing operations in dark environment.

10. The vision aiding apparatus integrated with the camera module and the light sensor according to claim 4, the apparatus further comprises a GPS module connected to the processing chip, for locating geographical location of the user and feeding back the geographical location to the processing chip, the processing chip confirming the surrounding environment of the user in combination with the geographical location and the data photographed by the camera module, to assist navigation for the user.
